(19) United States
(12) Patent Application Publication
(10) Pub. No.: US 2010/0190817 A1
(43) Pub. Date: Jul. 29, 2010

(54) LARGE SUBSTITUENT, NON-PHENOLIC AMINE OPIOIDS

(75) Inventor: Mark P. Wentland, Menands, NY (US)

Correspondence Address: HESLIN ROTHENBERG FARLEY & MESITI PC, 5 COLUMBIA CIRCLE, ALBANY, NY, 12203, US

(73) Assignee: RENSSELAER POLYTECHNIC INSTITUTE, Troy, NY (US)

(21) Appl. No.: 12/506,354

(22) Filed: Jul. 21, 2009

Related U.S. Application Data
(60) Provisional application No. 61/082,255, filed on Jul. 21, 2008.

(51) Int. Cl. A61K 31/439; C07D 489/00; C07D 221/28; C07D 221/26; A61P 25/36; A61P 25/00; A61P 1/12; A61P 3/04; A61P 11/00; A61P 25/30; A61P 25/32

(52) U.S. Cl. 514/295; 546/39; 546/44; 546/74; 546/97

(57) ABSTRACT

8-Substituted-2,6-methano-3-benzazocines of general structure

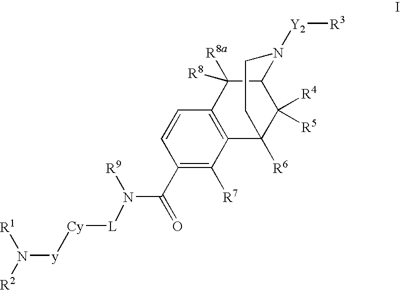

are useful as analgesics, anti-diarrheal agents, anticonvulsants, antitussives and anti-addiction medications.

### FEDERALLY SPONSORED RESEARCH

The following invention was made with Government support under contract number R01 DA12180 awarded by U.S. Dept of Health and Human Services. The Government has certain rights in this invention.

### FIELD OF THE INVENTION

The invention relates to opioid receptor binding compounds containing carboxamides that have large substitutents on the nitrogen of the carboxamide. The compounds are useful as analgesics, anti-diarrheal agents, anticonvulsants, anti-obesity agents, antitussives, anti-cocaine, and anti-addiction medications.

### BACKGROUND OF THE INVENTION

Opiates have been the subject of intense research since the isolation of morphine in 1805, and thousands of compounds having opiate or opiate-like activity have been identified. Many opioid receptor-interactive compounds including those used for producing analgesia (e.g., morphine) and those used for treating drug addiction (e.g., naltrexone and cyclazocine) in humans have limited utility due to poor oral bioavailability and a very rapid clearance rate from the body. This has been shown in many instances to be due to the presence of the 8-hydroxyl group (OH) of 2,6-methano-3-benzazocines, also known as benzomorphans [(e.g., cyclazocine and EKC (ethylketocyclazocine)] and the corresponding 3-OH group in morphinanes (e.g., morphine).

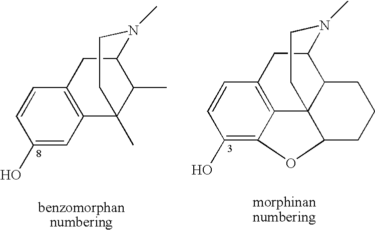

The high polarity of these hydroxyl groups retards oral absorption of the parent molecules. Furthermore, the 8-(or 3-)OH group is prone to sulfonation and glucuronidation (Phase II metabolism), both of which facilitate rapid excretion of the active compounds, leading to disadvantageously short half-lives for the active compounds. Until the publications of Wentland in 2001, the uniform experience in the art of the past seventy years had been that removal or replacement of the 8-(or 3-) OH group had led to pharmacologically inactive compounds.

U.S. Pat. No. 6,784,187 (to Wentland) disclosed that the phenolic OH of opioids could be replaced by CONH₂. In the cyclazocine series of opioids, it was shown that 8-carboxamidocyclazocine (8-CAC) had high affinity for μ and κ opioid receptors. In studies in vivo, 8-CAC showed high antinociception activity and a much longer duration of action than cyclazocine (15 h vs. 2 h) when both were dosed at 1 mg/kg ip in mice. Preliminary structure-activity relationship studies for 8-CAC revealed that mono-substitution of the carboxamide nitrogen with methyl or phenyl reduced binding affinity for guinea pig μ receptors 75- and 2313-fold, respectively whereas dimethylation of the carboxamide group reduced binding affinity 9375-fold. The finding that substitution of the carboxamide nitrogen had such a detrimental effect suggested that the NH₂ of the amide was critical to opioid binding.

### SUMMARY OF THE INVENTION

We have now found that the nitrogen of the carboxamide can be substituted with fairly large and relatively non-polar groups, and that such compounds exhibit excellent opioid binding and, presumably, good metabolic stability. The compounds of the invention are therefore useful as analgesics, anti-pruritics, anti-diarrheal agents, anticonvulsants, antitussives, anorexics and as treatments for hyperalgesia, drug addiction, respiratory depression, dyskinesia, pain (including neuropathic pain), irritable bowel syndrome and gastrointestinal motility disorders. Drug addiction, as used herein, includes alcohol and nicotine addiction. There is evidence in the literature that the compounds may also be useful as immunosuppressants and antiinflammatories and for reducing ischemic damage (and cardioprotection), for improving learning and memory, and for treating urinary incontinence.

In one aspect, the invention relates to compounds of formula I:

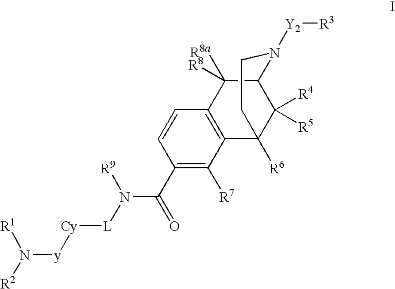

wherein
R¹ and R² are each independently chosen from hydrogen, optionally substituted lower alkyl, optionally substituted alkenyl, optionally substituted alkynyl, optionally substituted aryl, —COR¹⁰, —SO₂R¹⁰, —CONR¹⁰R¹¹, —C(═S)R¹⁰, C(═NOR¹¹)R¹⁰, C(═NR¹⁰)R¹¹ and —SO₂NR¹⁰R¹¹;
or, taken together with the nitrogen to which they are attached, R¹ and R² may form from one to three rings, said rings having optional additional substitution;
R³ is chosen from hydrogen, C₁-C₈ hydrocarbon, heterocyclyl, aryl and hydroxyalkyl;
R⁴ is chosen from hydrogen, hydroxyl, amino, lower alkoxy, C₁-C₂₀ alkyl and C₁-C₂₀ alkyl substituted with hydroxyl or carbonyl;
R⁵ is lower alkyl;
R⁶ is lower alkyl;
R⁷ is chosen from hydrogen, NR¹⁰R¹¹ and —OR¹⁶; or together R⁴, R⁵, R⁶ and R⁷ may form from one to three rings, said rings having optional additional substitution;
R⁸ and R^{8a} are both hydrogen or taken together R⁸ and R^{8a} are ═O;
R⁹ is chosen from hydrogen and lower alkyl;
R¹⁰ and R¹¹ are each independently hydrogen, optionally substituted lower alkyl, optionally substituted alkenyl, optionally substituted alkynyl, optionally substituted aryl, hydroxyl, amino or optionally substituted lower alkoxy;
y is —(C(R¹⁰)(R¹¹))p- or a direct bond, wherein p is 0, 1, 2, 3, 4, 5, 6, or 7;
Y₂ is a direct bond or —(C(R¹⁰)(R¹¹))q- wherein q is 0, 1, 2, 3, 4 or 5;
L is a direct bond or —(C(R¹⁰)(R¹¹))q-; and
Cy is Ar¹—B—Ar², wherein
   Ar¹ is absent, or an aryl or heteroaryl radical having from 1 to 4 N, O and/or S atoms, which may be unsubstituted or mono-, di- or trisubstituted by halogen, lower alkyl, alkenyl, alkynyl, cycloalkyl, —OR¹⁰, —NR¹⁰R¹¹, —CN, —COR¹⁰ or —COOR¹⁰;
   B is a direct bond, —O—, —NR¹⁰, —SO₂, or —(C(R¹⁰)R¹¹)s-, wherein s is 0, 1, 2, 3, 4 or 5; and

Ar² is aryl or heteroaryl radical having from 1 to 4 N, O and/or S atoms, which may be unsubstituted or mono-, di- or trisubstituted by halogen, lower alkyl, alkenyl, alkynyl, cycloalkyl, —OR¹⁰, —NR¹⁰R¹¹, —CN, —COR¹⁰ or —COOR¹⁰.

In another aspect, the invention relates to a method for preparing a second compound that interacts with an opioid receptor when a first compound that interacts with an opioid receptor is known, said first compound containing a phenolic hydroxyl, said method comprising converting said phenolic hydroxyl to a residue of formula:

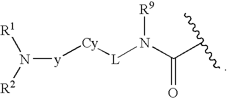

In some embodiments, the residue

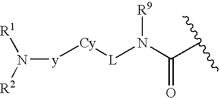

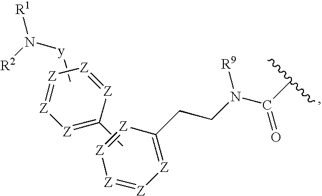

wherein Z is CR¹⁰ or N, with the proviso that, at the points of attachment of the NR¹R²y group to the distal aromatic ring and of the distal aromatic ring to the proximal aromatic ring, Z must be C.

In another aspect, the invention relates to a pharmaceutical formulation comprising a compound of formula I and a pharmaceutically acceptable carrier.

In another aspect, the invention relates to a method of preventing or treating a condition or disease associated with binding opioid receptors in a patient in need thereof, comprising the step of administering to said patient a composition comprising an effective amount of a compound of formula I. In some embodiments, the disease or condition to be treated or prevented is pain, pruritis, diarrhea, irritable bowel syndrome, gastrointestinal motility disorder, obesity, respiratory depression, convulsions, coughing, hyperalgesia and drug addiction. In further embodiments, drug addiction encompasses heroin, cocaine, nicotine or alcohol addiction. In other embodiments, the condition is pain and the composition further comprises an effective amount of an opioid.

### DETAILED DESCRIPTION OF THE INVENTION

From many years of SAR studies, it is known that the hydroxyl of morphinans and benzomorphans interacts with a specific site in the opiate receptor. We have now surprisingly found that the hydroxyl can be replaced with a very large carboxamide residue. A fairly wide range of secondary carboxamides exhibits binding in the desired range below 25 nanomolar.

Since phenolic hydroxyls of benzomorphans and morphinans can be chemically converted to carboxamides by a simple, flexible and convenient route described in U.S. Pat. Nos. 6,784,187 and 7,057,035, the door is opened to a whole family of new therapeutic agents, many of which derive directly from the application of the principles set forth herein to known therapeutic agents that rely on opioid binding for their activity. Moreover, since the receptor seems to tolerate some variation in Q, one may contemplate further modulating receptor specificity, affinity and tissue distribution by varying the properties of the aryl substituents.

In one aspect the invention relates to compounds of formula I:

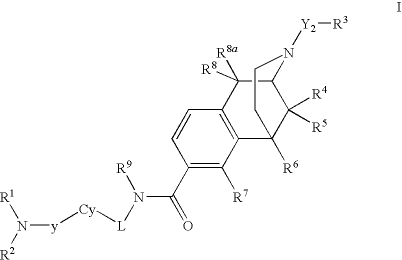

wherein
R¹ and R² are each independently chosen from hydrogen, optionally substituted lower alkyl, optionally substituted alkenyl, optionally substituted alkynyl, optionally substituted aryl, —COR¹⁰, —SO₂R¹⁰, —CONR¹⁰R¹¹, —C(═S)R¹⁰, —C(═NOR¹¹)R¹⁰, C(═NR¹⁰)R¹¹ and —SO₂NR¹⁰R¹¹;
or, taken together with the nitrogen to which they are attached, R¹ and R² may form from one to three rings, said rings having optional additional substitution;
R³ is chosen from hydrogen, C₁-C₈ hydrocarbon, heterocyclyl, aryl and hydroxyalkyl;
R⁴ is chosen from hydrogen, hydroxyl, amino, lower alkoxy, C₁-C₂₀ alkyl and C₁-C₂₀ alkyl substituted with hydroxyl or carbonyl;
R⁵ is lower alkyl;
R⁶ is lower alkyl;
R⁷ is chosen from hydrogen, NR¹⁰R¹¹ and —OR¹⁰, or
together R⁴, R⁵, R⁶ and R⁷ may form from one to three rings, said rings having optional additional substitution;
R⁸ and R^{8a} are both hydrogen or taken together R⁸ and R^{8a} are ═O;
R⁹ is chosen from hydrogen and lower alkyl;
R¹⁰ and R¹¹ are each independently hydrogen, optionally substituted lower alkyl, optionally substituted alkenyl, optionally substituted alkynyl, optionally substituted aryl, hydroxyl, amino or optionally substituted lower alkoxy;
y is —(C(R¹⁰)(R¹¹))p- or a direct bond, wherein p is 0, 1, 2, 3, 4, 5, 6, or 7;
Y₂ is a direct bond or —(C(R¹⁰)(R¹¹))q-, wherein q is 0, 1, 2, 3, 4 or 5;
L is a direct bond or —(C(R¹⁰)(R¹¹))q-; and
Cy is Ar¹—B—Ar², wherein
   Ar¹ is absent, or an aryl or heteroaryl radical having from 1 to 4 N, O and/or S atoms, which may be unsubstituted or mono-, di- or trisubstituted by halogen, lower alkyl, alkenyl, alkynyl, cycloalkyl, —OR¹⁰, —NR¹⁰R¹¹, —CN, —COR¹⁰ or —COOR¹⁰;
   B is a direct bond, —O—, —NR¹⁰, —SO₂, or —(C(R¹⁰)(R¹¹)s-, wherein s is 0, 1, 2, 3, 4 or 5; and
   Ar² is aryl or heteroaryl radical having from 1 to 4 N, O and/or S atoms, which may be unsubstituted or mono-, di- or trisubstituted by halogen, lower alkyl, alkenyl, alkynyl, cycloalkyl, —OR¹⁰, —NR¹⁰R¹¹, —CN, —COR¹⁰ or —COOR¹⁰.

Subclasses of the foregoing structure include:

II. 2,6-methano-3-benzazocines of the structure shown above, in which R⁴, R⁵, R⁶ and R⁷ do not form additional rings:

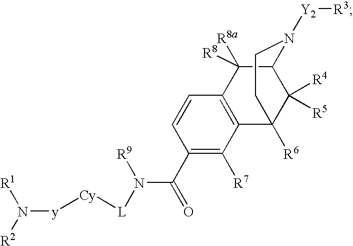

III. morphinans in which R⁵ and R⁶ form one ring:

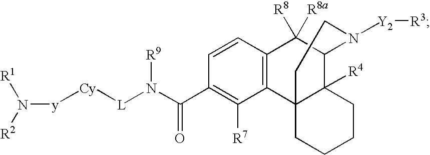

IV. morphinans in which R⁵, R⁶ and R⁷ form two rings:

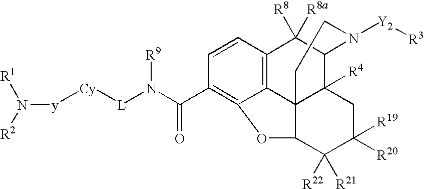

wherein

R¹⁹ is hydrogen or lower alkyl;

R²⁰ is chosen from hydrogen, lower alkyl and hydroxy(lower alkyl); or together, R¹⁹ and R²⁰ form a spiro-fused carbocycle of 5 to 10 carbons;

R²¹ is hydrogen;

R²² is chosen from hydroxyl, lower alkoxy and —NR¹³R¹⁴; or together, R²¹ and R²² form a carbonyl or a vinyl substituent;

R¹³ is hydrogen or optionally substituted lower alkoxy; and

R¹⁴ is hydrogen, optionally substituted lower alkoxy, acyl or fumarate.

V. morphinans wherein R⁴ and R²¹ form an additional sixth ring, which may be saturated:

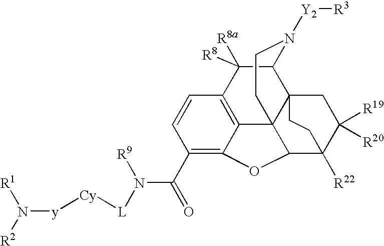

or unsaturated:

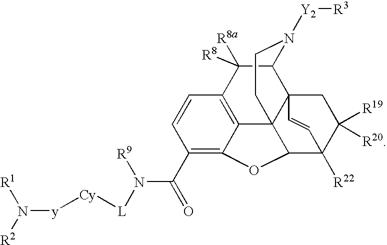

In some embodiments, examples of Cy include, but are not limited to:

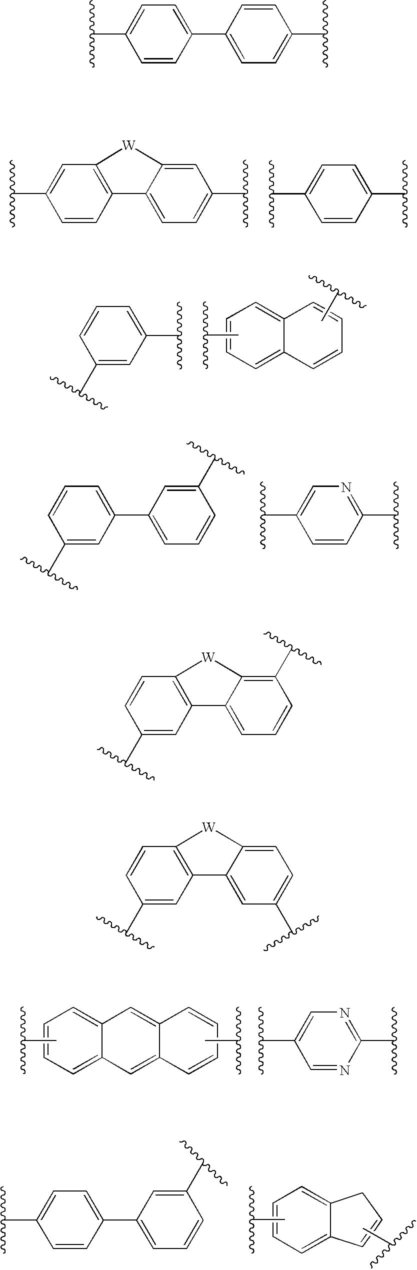

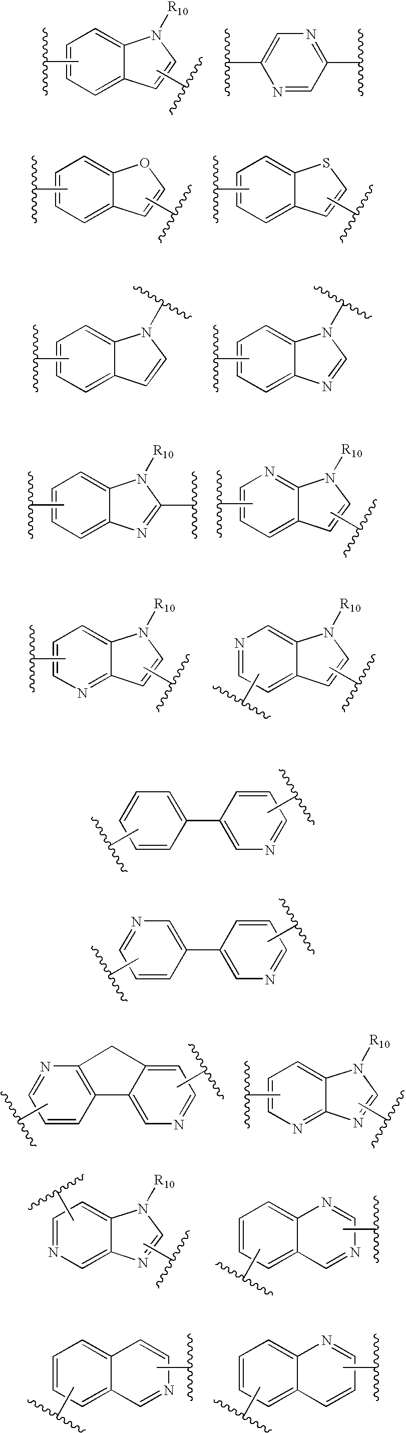

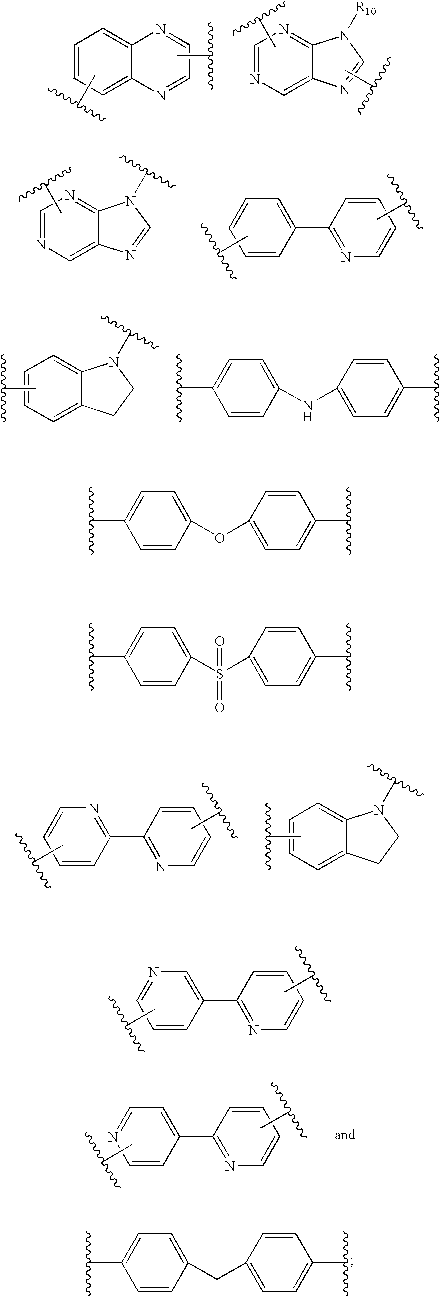

wherein W is selected from [C(R⁹)₂]ₙ, CR⁸R^{8a}, O, NR⁹, S and CR⁹═CR⁹; and

n is 1, 2, 3, 4 or 5.

In some embodiments, the invention relates to compounds of formula II:

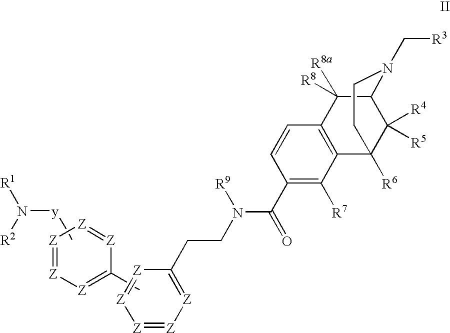

In some embodiments, Z is N. In still other embodiments, Z is CR¹⁰. In further embodiments, R¹⁰ is hydrogen. In other embodiments, R¹⁰ is optionally substituted lower alkyl or optionally substituted lower alkoxy. In further embodiments, R¹⁰ is methyl.

In some embodiments, R¹ and R² are each hydrogen. In other embodiments, R¹ is hydrogen and R² is optionally substituted lower alkyl. In still other embodiments, R¹ and R² are each optionally substituted lower alkyl. In some of these embodiments, R¹ and R² are each methyl. In yet other embodiments, R¹ is hydrogen, R² is —COR¹⁰, and R¹⁰, is optionally substituted lower alkoxy. In some of these embodiments, R¹⁰ is tert-butoxy. In still other embodiments, R² is, together with the nitrogen to which it is attached, fluorenylmethyl carbamate, tert-butyl carbamate, benzyl carbamate, acetamide, trifluoroacetamide, benzylamine, triphenylmethylamine or toluenesulfonamide. In further embodiments, R¹ and R² may form, together with the nitrogen to which they are attached, from one to three rings, said rings having optional additional substitution.

In some embodiments, R³ is hydrogen. In other embodiments, R³ is heterocyclyl. In still other embodiments, R³ is hydroxyalkyl. In yet other embodiments, R³ is C₁-C₈ hydrocarbon. In further embodiments, R³ is cyclopropyl or cyclobutyl.

In some embodiments, R⁴ is hydrogen. In other embodiments, R⁴ is hydroxyl or amino. In still other embodiments, R⁴ is lower alkoxy. In yet other embodiments, R⁴ is C₁-C₂₀ alkyl or C₁-C₂₀ alkyl substituted with hydroxyl or carbonyl. In further embodiments, R⁴ is methyl or ethyl.

R⁵ is lower alkyl. In some embodiments, R⁵ is methyl.

R⁶ is lower alkyl. In some embodiments, R⁶ is methyl.

In some embodiments, R⁷ is hydrogen. In other embodiments, R⁷ is —OR¹⁰. In further embodiments, R⁷ is hydroxyl. In still other embodiments, R⁷ is NR¹⁰R¹¹. In further embodiments, R⁷ is NH₂, NHCH₃ or NH(CH₃)₂.

In some embodiments, R⁴, R⁵, R⁶ and R⁷ together may form from one to three rings, said rings having optional additional substitution. Some representative examples are shown above in subgenera III, IV and V.

In an embodiment of the invention, R⁸ and R^{8a} are both hydrogen. In another embodiment, R⁸ and R^{8a} are taken together to form ═O.

In some embodiments, R⁹ is hydrogen. In other embodiments, R⁹ is lower alkyl.

In some embodiments, R¹⁰ and R¹¹ are each independently hydrogen. In other embodiments, R¹⁰ is optionally substituted lower alkoxy and R¹¹ is hydrogen or methyl. In still other embodiments, R¹⁰ is optionally substituted lower alkyl and R¹¹ is hydrogen or methyl. In yet other embodiments, R¹⁰ is optionally substituted aryl and R¹¹ is hydrogen or methyl. In yet other embodiments, R¹⁰ is hydroxyl or amino and R¹¹ is hydrogen or methyl.

In an embodiment of the invention, y is CH₂. In another embodiment, y is a direct bond.

In some embodiments, Z is CH. In other embodiments, Z is N. At the points of attachment of the NR¹R²y group to the distal aromatic ring and of the distal aromatic ring to the proximal aromatic ring, Z must be C.

In some embodiments of the invention, formula II has the orientation below:

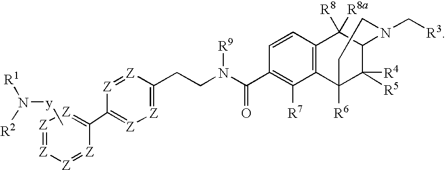

The residue shown here

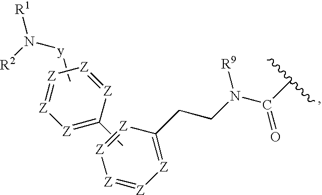

will hereinafter be sometimes referred to as Q.

In certain embodiments of the invention, the aromatic rings of Q have a para orientation:

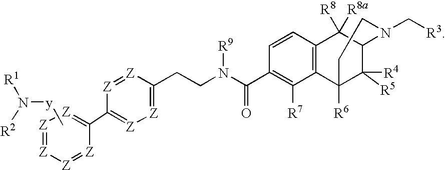

In still other embodiments, each Z is equal to carbon

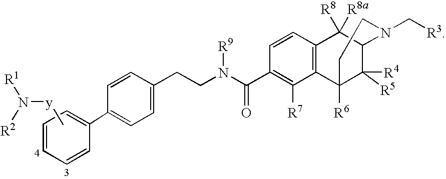

In certain embodiments of formula I and formula II, R⁸, R^{8a} and R⁹ are each hydrogen, R⁵ is methyl and R⁶ is methyl or ethyl. In some of these embodiments, R⁴ is hydrogen. In other embodiments, R⁴ is 3-oxo-5-cyclopentyl-1-pentanyl. In some of these embodiments, R³ is cyclopropyl. In other embodiments, R³ is hydroxycyclopropyl. In other embodiments, R³ is cyclobutyl. In still other embodiments, R³ is hydrogen. In yet other embodiments, R³ is phenyl, furanyl or tetrahydrofuranyl. In further embodiments, R³ is vinyl or dimethylvinyl.

In some embodiments of the invention, -yNR¹R² is attached in the para orientation (the 4-position):

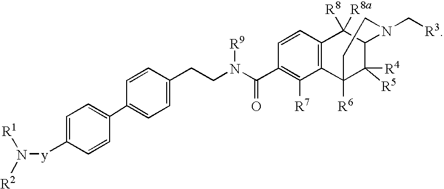

In some of these embodiments, y is a direct bond. In yet other embodiments, R¹ and R² are each equal to lower alkyl. In some embodiments, R¹ and R² are each selected from hydrogen and methyl. In further embodiments, R¹ and R² are both methyl. In other embodiments, R¹ is hydrogen and R² is substituted alkyl. For instance, R² could be triphenylmethyl or benzyl. In other embodiments, R¹ is hydrogen and R² is —SO₂R¹⁰. In some of these embodiments, R¹⁰ is optionally substituted aryl, for instance, toluene. In still other embodiments, R¹ is hydrogen and R² is —COR¹⁰. In some of these embodiments, R¹⁰ is optionally substituted alkoxy, for instance, fluorenylmethoxy, t-butoxy, or benzyloxy. In other of these embodiments, R¹⁰ is optionally substituted alkyl, for instance, methyl or trifluoromethyl. In some of these embodiments, R² is, together with the nitrogen to which it is attached, fluorenylmethyl carbamate, tert-butyl carbamate, benzyl carbamate, acetamide, trifluoroacetamide, benzylamine, triphenylmethylamine or toluenesulfonamide. In still other embodiments, —NR¹R² together form from one to three optionally substituted rings. One example is phthalimide.

In some of these embodiments, y is —CH₂. In yet other embodiments, R¹ and R² are each equal to lower alkyl. In some embodiments, R¹ and R² are each selected from hydrogen and methyl. In further embodiments, R¹ and R² are both methyl. In other embodiments, R¹ is hydrogen and R² is substituted alkyl. For instance, R² could be triphenylmethyl or benzyl. In other embodiments, R¹ is hydrogen and R² is —SO₂R¹⁰. In some of these embodiments, R¹⁰ is optionally substituted aryl, for instance, toluene. In still other embodiments, R¹ is hydrogen and R² is —COR¹⁰. In some of these embodiments, R¹⁰ is optionally substituted alkoxy, for instance, fluorenylmethoxy, t-butoxy, or benzyloxy. In other of these embodiments, R¹⁰ is optionally substituted alkyl, for instance, methyl or trifluoromethyl. In some of these embodiments, R² is, together with the nitrogen to which it is attached, fluorenylmethyl carbamate, tert-butyl carbamate, benzyl carbamate, acetamide, trifluoroacetamide, benzylamine, triphenylmethylamine or toluenesulfonamide. In still other embodiments, —NR¹R² together form from one to three optionally substituted rings. One example is phthalimide.

In some embodiments of the invention, -yNR¹R² is attached in the meta orientation (the 3-position). In some of these embodiments, y is a direct bond. In other embodiments, R¹ and R² are each selected from hydrogen and methyl. In other embodiments, R¹ is hydrogen and R² is substituted alkyl. For instance, R² could be triphenylmethyl or benzyl. In other embodiments, R¹ is hydrogen and R² is —SO₂R¹⁰. In some of these embodiments, R¹⁰ is optionally substituted aryl, for instance, toluene. In still other embodiments, R¹ is hydrogen and R² is —COR¹⁰. In some of these embodiments, R¹⁰ is optionally substituted alkoxy, for instance, fluorenylmethoxy, t-butoxy, or benzyloxy. In other of these embodiments, R¹⁰ is optionally substituted alkyl, for instance, methyl or trifluoromethyl. In some of these embodiments, R² is, together with the nitrogen to which it is attached, fluorenylmethyl carbamate, tert-butyl carbamate, benzyl carbamate, acetamide, trifluoroacetamide, benzylamine, triphenylmethylamine or toluenesulfonamide. In still other embodiments, —NR¹R² together form from one to three optionally substituted rings. One example is phthalimide.

In some embodiments, R⁵ and R⁶ together form one ring:

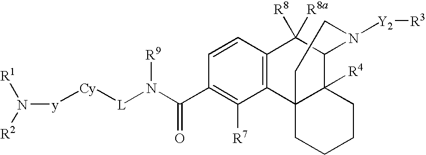

In some of these embodiments, R⁴, R⁸ and R^{8a} are each hydrogen. In other embodiments, R⁸ and R^{8a} are each hydrogen and R⁴ is hydroxyl. In still other embodiments, R⁴ is amino. In some of these embodiments, R³ is hydrogen. In other embodiments, R³ is cyclopropyl or cyclobutyl. In still other embodiments, R³ is vinyl. In yet other embodiments, R³ is tetrahydrofuranyl. In some embodiments, the compounds are of formula

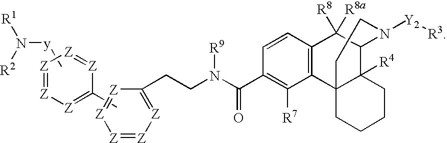

In some embodiments, together R⁵, R⁶ and R⁷ form two rings, having the structure:

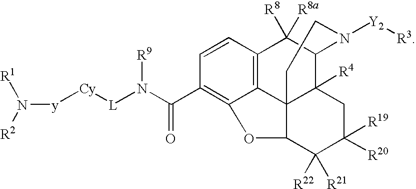

In these embodiments, R¹⁹ is hydrogen or lower alkyl; and R²¹ is hydrogen. In some of these embodiments, R²⁰ is chosen from hydrogen, lower alkyl and hydroxy(lower alkyl) In other embodiments, R¹⁹ and R²⁰ together form a spiro-fused carbocycle of 5 to 10 carbons. In yet other embodiments, R²² is chosen from hydroxy, lower alkoxy and —NR¹³R¹⁴. In still other embodiments, R¹³ is hydrogen or optionally substituted lower alkoxy. In yet other embodiments, R¹⁴ is hydrogen, optionally substituted lower alkoxy, acyl or fumarate.

In still other embodiments, R²¹ and R²² together form a carbonyl or a vinyl substituent. In some embodiments, the compounds are of formula

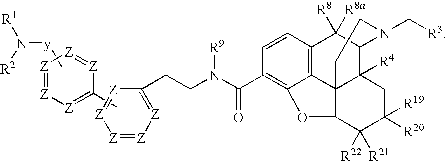

In other embodiments, together, R⁴ and R²¹ form a sixth ring exemplified below:

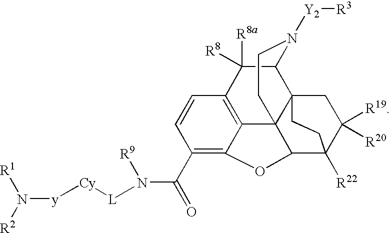

In some of these embodiments, the compounds are of formula

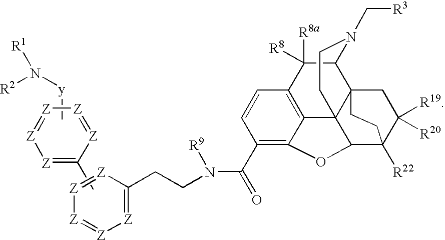

In another embodiment, R⁴ and R²¹ form a sixth ring exemplified by:

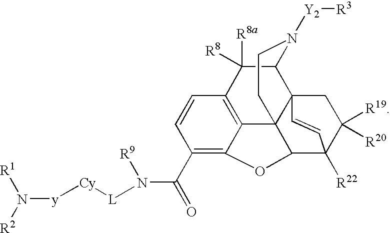

In some embodiments, the compounds are of formula

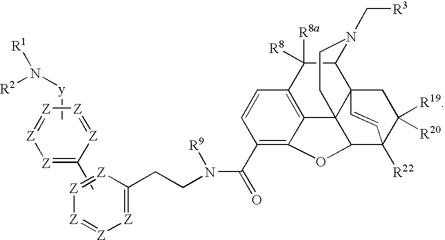

In some of these embodiments, R¹⁹ is hydrogen; R²⁰ is hydroxy(lower alkyl); and R²² is lower alkoxy.

In another aspect, the invention relates to a method for preparing a second compound that interacts with an opioid receptor when a first compound that interacts with an opioid receptor is known. When the first compound contains a phenolic hydroxyl, the method comprises converting the phenolic hydroxyl to a residue of structure:

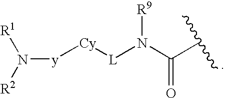

In some embodiments, the residue is

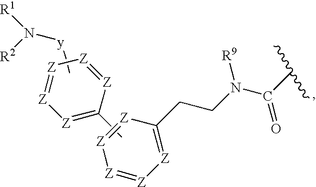

which will be sometimes referred to as Q.

It is known in the art that compounds that are μ, δ and κ agonists exhibit analgesic activity; compounds that are selective μ agonists exhibit anti-diarrheal activity and are useful in treating dyskinesia; μ antagonists and κ agonists are useful in treating heroin, cocaine, alcohol and nicotine addiction; κ agonists are also anti-pruritic agents and are useful in treating hyperalgesia. Recently it has been found [Peterson et al. Biochem. Pharmacol. 61, 1141-1151 (2001)] that κ agonists are also useful in treating retroviral infections. In general, the dextrorotatory isomers of morphinans of type III above are useful as antitussives and anticonvulsants.

Opioid receptor ligands having known high affinity are shown in the following charts. Replacement of OH with the

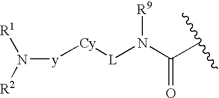

residue or with Q in these compounds produces compounds that exhibit similar activity and better bioavailability.

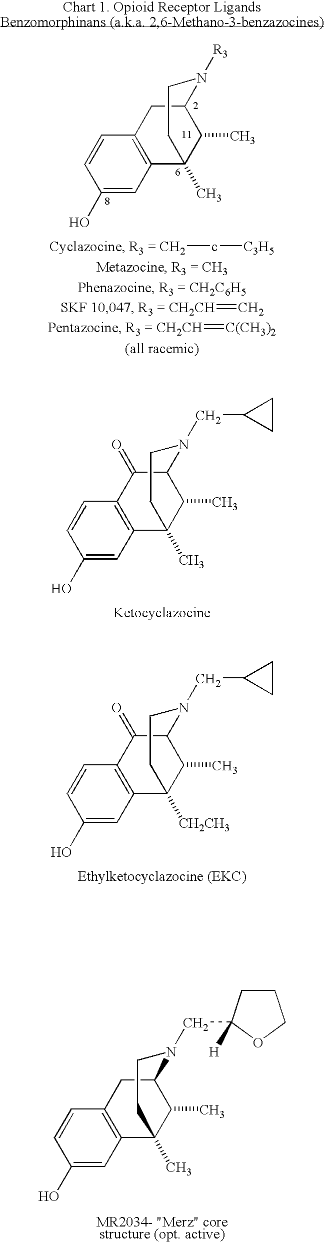

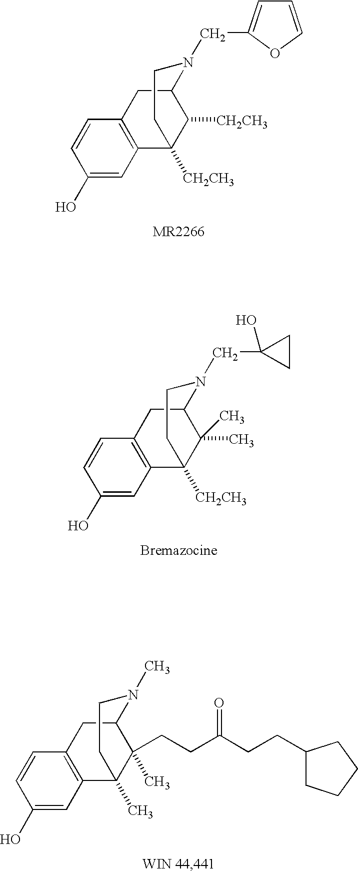

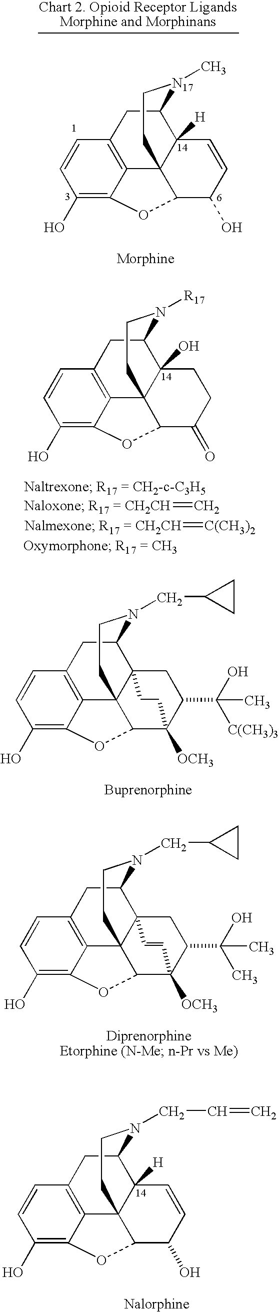

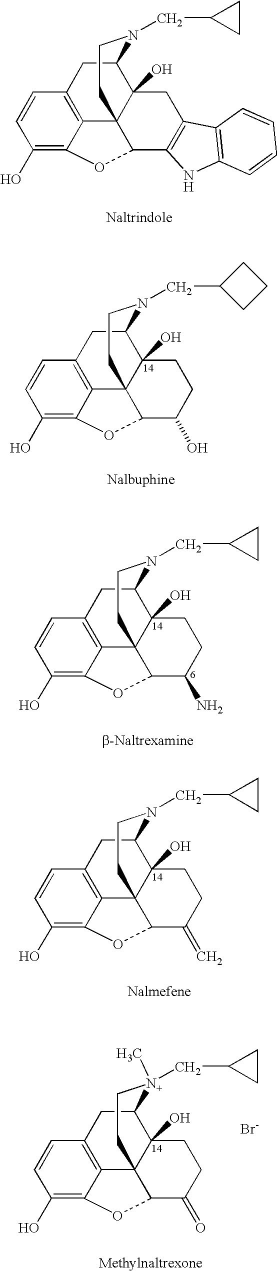

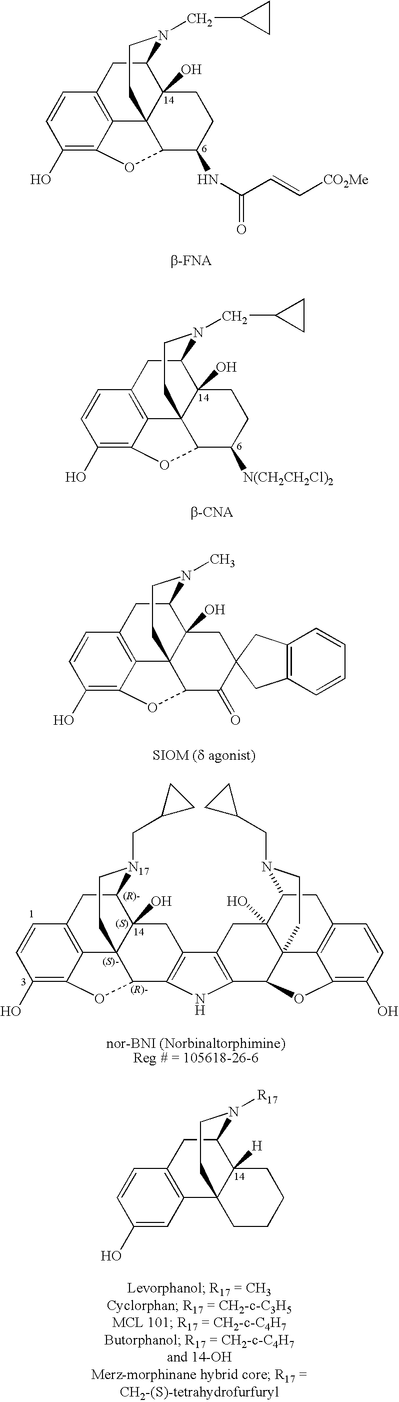

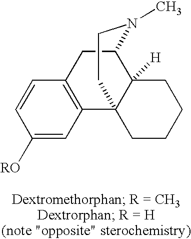

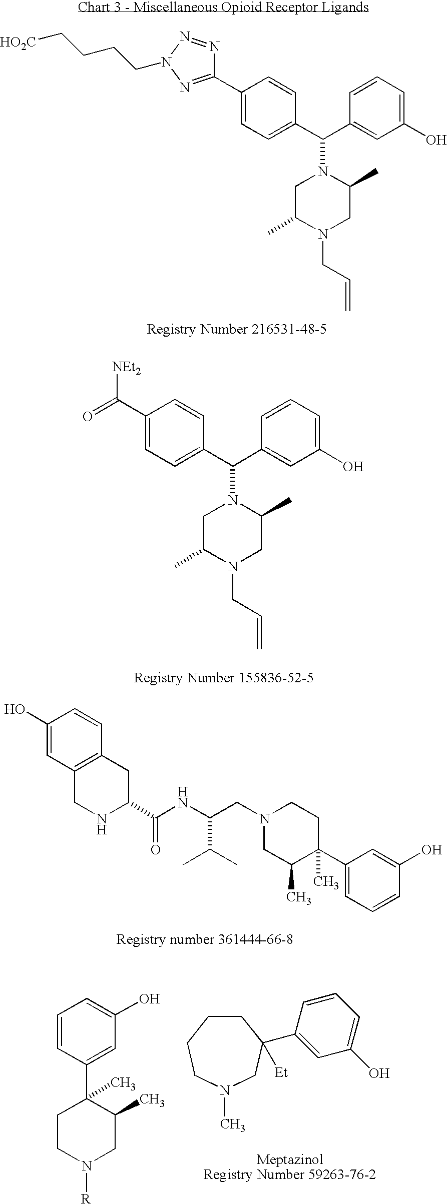

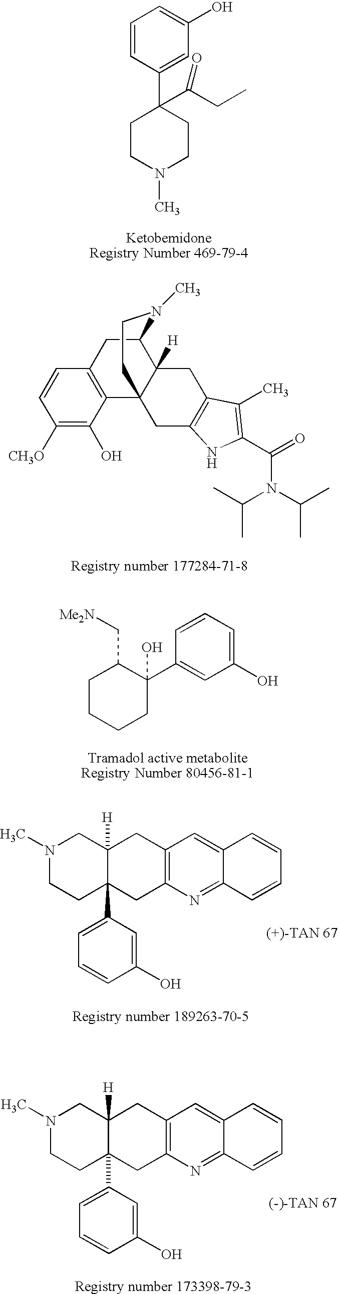

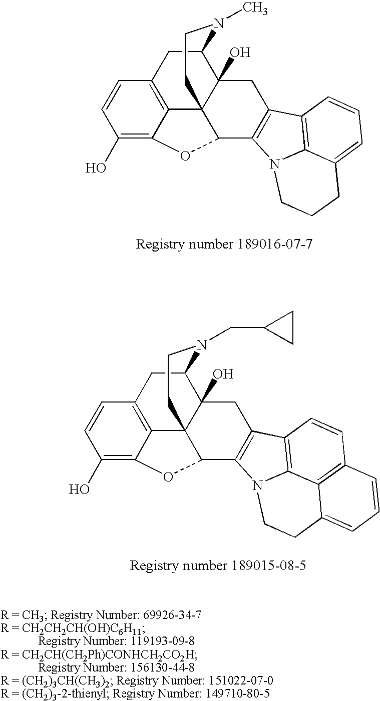

Other opioid receptor ligands are described in Aldrich, J. V. “Analgesics” in Burger's Medicinal Chemistry and Drug Discovery, M. E. Wolff ed., John Wiley & Sons 1996, pages 321-44, the disclosures of which are incorporated herein by reference. In all but two of the foregoing compounds, there is a single phenolic OH that is to be replaced by the

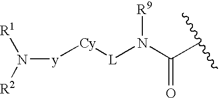

residue or by Q according to the present invention. In norbinaltorphimine and 361444-66-8, there are two phenolic OH's, either or both of which are replaced by the

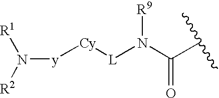

residue or by Q.

Binding assays used to screen compounds are similar to those previously reported by Neumeyer et al., Design and Synthesis of Novel Dimeric Morphinan Ligands for κ and μ Opioid Receptors. J. Med. Chem. 2003, 46, 5162. Membrane protein from CHO cells that stably expressed one type of the human opioid receptor were incubated with 12 different concentrations of the compound in the presence of either 1 nM [³H]U69,593¹⁰ (κ), 0.25 nM [³H]DAMGO¹¹ (μ) or 0.2 nM [³H]naltrindole¹² (δ) in a final volume of 1 mL of 50 mM Tris-HCl, pH 7.5 at 25° C. Incubation times of 60 min were used for [³H]U69,593 and [³H]DAMGO. Because of a slower association of [³H]naltrindole with the receptor, a 3 h incubation was used with this radioligand. Samples incubated with [³H]naltrindole also contained 10 mM MgCl₂ and 0.5 mM phenylmethylsulfonyl fluoride. Nonspecific binding was measured by inclusion of 10 μM naloxone. The binding was terminated by filtering the samples through Schleicher & Schuell No. 32 glass fiber filters using a Brandel 48-well cell harvester. The filters were subsequently washed three times with 3 mL of cold 50 mM Tris-HCl, pH 7.5, and were counted in 2 mL Ecoscint A scintillation fluid. For [³H]naltrindole and [³H]U69,593 binding, the filters were soaked in 0.1% polyethylenimine for at least 60 min before use. IC₅₀ values were-calculated by least squares fit to a logarithm-probit analysis. Kᵢ values of unlabeled compounds were calculated from the equation Kᵢ=(IC₅₀)/1+S where S=(concentration of radioligand)/(K_{d} of radioligand).¹³ Data are the mean±SEM from at least three experiments performed in triplicate.

[³⁵S]GTPγS Binding Assays. In a final volume of 0.5 mL, 12 different concentrations of each test compound were incubated with 15 μg (κ), 10 μg (δ) or 7.5 μg (μ) of CHO cell membranes that stably expressed either the human κ, δ or μ opioid receptor. The assay buffer consisted of 50 mM Tris-HCl, pH 7.4, 3 mM MgCl₂, 0.2 mM EGTA, 3 μM GDP, and 100 mM NaCl. The final concentration of [³⁵S]GTPγS was 0.080 nM. Nonspecific binding was measured by inclusion of 10 μM GTPγS. Binding was initiated by the addition of the membranes. After an incubation of 60 min at 30° C., the samples were filtered through Schleicher & Schuell No. 32 glass fiber filters. The filters were washed three times with cold 50 mM Tris-HCl, pH 7.5, and were counted in 2 mL of Ecoscint scintillation fluid. Data are the mean Eₘₐₓ and EC₅₀ values±S.E.M. from at least three separate experiments, performed in triplicate. For calculation of the Eₘₐₓ values, the basal [³⁵S]GTPγS binding was set at 0%. To determine antagonist activity of a compound at the μ opioid receptors, CHO membranes expressing the μ opioid receptor, were incubated with 12 different concentrations of the compound in the presence of 200 nM of the μ agonist DAMGO. To determine antagonist activity of a compound at the κ opioid receptors, CHO membranes expressing the κ opioid receptor, were incubated with the compound in the presence of 100 nM of the κ agonist U50,488. To determine if a compound was an antagonist at δ receptors, CHO membranes expressing the δ receptor were incubated with 12 different concentrations of the test compound in the presence of 10 nM of the δ-selective agonist SNC 80.

### Examples

### Cyclazocine Subseries

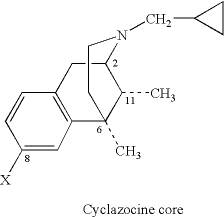

| | | Kᵢ (nM) | | |
|---|---|---|---|---|
| Example No. | X | [³H]DAMGO (μ) | [³H]Naltrindole (δ) | [³H]U69,593 (κ) |
| MV-E-126 | CONH(CH₂)₂(4-C₆H₄-4-(CH₃)₂NC₆H₄) | 0.087 ± 0.0077 | 1.4 ± 0.071 | 0.76 ± 0.12 |
| SJJ-B-074c | CONH(CH₂)₂(4-C₆H₄-3-(CH₃)₂NC₆H₄) | 0.18 ± 0.055 | 2.5 ± 0.17 | 0.26 ± 0.022 |
| SJJ-B-112g | CONH(CH₂)₂(4-C₆H₄-4-NH₂C₆H₄) | 0.0014 ± 0.00010 | 1.5 ± 0.078 | 0.39 ± 0.0085 |
| SJJ-C-027b | CONH(CH₂)₂(4-C₆H₄-4-BocNHC₆H₄) | 0.32 ± 0.015 | 3.1 ± 0.34 | 3.4 ± 0.32 |
| SJJ-C-013b | CONH(CH₂)₂(4-C₆H₄-4-(CH₃)₂NCH₂C₆H₄) | 0.094 ± 0.0054 | 3.7 ± 0.15 | 1.9 ± 0.014 |

Kᵢ (nM ± S.E.)

TABLE 3
Other Opioid Parents

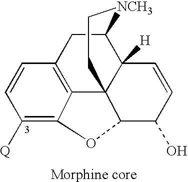

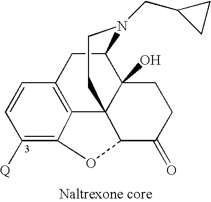

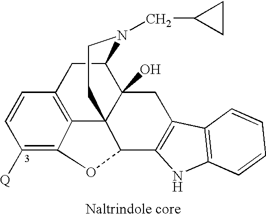

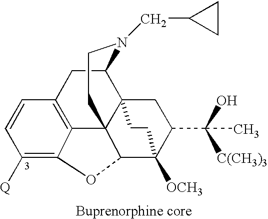

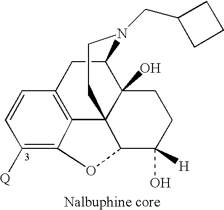

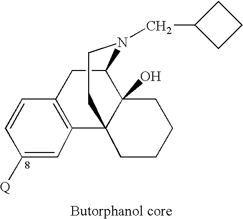

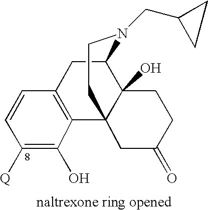

Antinociceptive activity is evaluated by the method described in Jiang et al. [J. Pharmacol. Exp. Ther. 264, 1021-1027 (1993), page 1022]. The ED₅₀.s of compounds of the invention are expected to be under 100 nmol in the mouse acetic acid writhing test when administered i.c.v., and an increase in the duration of action is expected for compounds of the invention compared to their “parents” when given by i.p. administration.

### DEFINITIONS

Throughout this specification the terms and substituents retain their definitions.

Alkyl is intended to include linear, branched, or cyclic hydrocarbon structures and combinations thereof. A combination would be, for example, cyclopropylmethyl. Lower alkyl refers to alkyl groups of from 1 to 6 carbon atoms. Examples of lower alkyl groups include methyl, ethyl, propyl, isopropyl, cyclopropyl, butyl, s-and t-butyl, cyclobutyl and the like. Preferred alkyl groups are those of C₂₀ or below. Cycloalkyl is a subset of alkyl and includes cyclic hydrocarbon groups of from 3 to 8 carbon atoms. Examples of cycloalkyl groups include c-propyl, c-butyl, c-pentyl, norbornyl and the like.

Alkoxy or alkoxyl refers to groups of from 1 to 8 carbon atoms of a straight, branched, or cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons.

Aryl and heteroaryl mean a 5- or 6-membered aromatic or heteroaromatic ring containing 0-3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S. The aromatic 6- to 14-membered carbocyclic rings include, e.g., benzene, naphthalene, indane, tetralin, and fluorene and the 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole. As used herein aryl and heteroaryl refer to residues in which one or more rings are aromatic, but not all need be.

Arylalkyl means an alkyl residue attached to an aryl ring. Examples are benzyl, phenethyl and the like. Heteroarylalkyl means an alkyl residue attached to a heteroaryl ring. Examples include, e.g., pyridinylmethyl, pyrimidinylethyl and the like.

C₁ to C₂₀ hydrocarbon means a linear, branched, or cyclic residue comprised of hydrogen and carbon as the only elemental constituents and includes alkyl, cycloalkyl, polycycloalkyl, alkenyl, alkynyl, aryl and combinations thereof. Examples include benzyl, phenethyl, cyclohexylmethyl, camphoryl and naphthylethyl.

Heterocycle means a cycloalkyl or aryl residue in which one to two of the carbons is replaced by a heteroatom such as oxygen, nitrogen or sulfur. Heteroaryls form a subset of heterocycles. Examples of heterocycles that fall within the scope of the invention include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like.

Substituted alkyl, aryl, cycloalkyl, heterocyclyl etc. refer to alkyl, aryl, cycloalkyl, or heterocyclyl wherein up to three H atoms in each residue are replaced with halogen, haloalkyl, alkyl, acyl, alkoxyalkyl, hydroxyloweralkyl, phenyl, heteroaryl, benzenesulfonyl, hydroxy, loweralkoxy, haloalkoxy, carboxy, carboalkoxy (also referred to as alkoxycarbonyl), alkoxycarbonylamino, carboxamido (also referred to as alkylaminocarbonyl), cyano, carbonyl, acetoxy, nitro, amino, alkylamino, dialkylamino, mercapto, alkylthio, sulfoxide, sulfone, sulfonylamino, acylamino, amidino, aryl, benzyl, heterocyclyl, phenoxy, benzyloxy, heteroaryloxy, hydroxyimino, alkoxyimino, oxaalkyl, aminosulfonyl, trityl, amidino, guanidino, ureido, and benzyloxy.

Virtually all of the compounds described herein contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, as well as their racemic and optically pure forms. In general it has been found that the levo isomer of morphinans and benzomorphans is the more potent antinociceptive agent, while the dextro isomer may be useful as an antitussive or antispasmodic agent. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

Some of the compounds of the invention are quaternary salts, i.e. cationic species. Therefore they will always be presented as salts, and the term “pharmaceutically acceptable salt” refers to salts whose counter ion (anion) derives from pharmaceutically acceptable non-toxic acids including inorganic acids, organic acids and water (which formally furnishes the hydroxide anion). Suitable pharmaceutically acceptable anions for the compounds of the present invention include hydroxide, acetate, benzenesulfonate (besylate), benzoate, bicarbonate, bisulfate, carbonate, camphorsulfonate, citrate, ethanesulfonate, fumarate, gluconate, glutamate, glycolate, bromide, chloride, isethionate, lactate, maleate, malate, mandelate, methanesulfonate, mucate, nitrate, pamoate, pantothenate, phosphate, succinate, sulfate, tartrate, trifluoroacetate, p-toluenesulfonate, acetamidobenzoate, adipate, alginate, aminosalicylate, anhydromethylenecitrate, ascorbate, aspartate, calcium edetate, camphorate, camsylate, caprate, caproate, caprylate, cinnamate, cyclamate, dichloroacetate, edetate (EDTA), edisylate, embonate, estolate, esylate, fluoride, formate, gentisate, gluceptate, glucuronate, glycerophosphate, glycolate, glycollylarsanilate, hexylresorcinate, hippurate, hydroxynaphthoate, iodide, lactobionate, malonate, mesylate, napadisylate, napsylate, nicotinate, oleate, orotate, oxalate, oxoglutarate, palmitate, pectinate, pectinate polymer, phenylethylbarbiturate, picrate, pidolate, propionate, rhodanide, salicylate, sebacate, stearate, tannate, theoclate, tosylate and the like. The desired salt may be obtained by ion exchange of whatever counter ion is obtained in the synthesis of the quat. These methods are well known to persons of skill. Although pharmaceutically acceptable counter ions will be preferred for preparing pharmaceutical formulations, other anions are quite acceptable as synthetic intermediates. Thus X may be pharmaceutically undesirable anions, such as iodide, oxalate, trifluoromethanesulfonate and the like, when such salts are chemical intermediates. When the compounds of the invention are bisquats, one may employ as counter ions either two monoanionic species (e.g. Cl₂) or a single dianionic species (e.g. fumarate). Similarly, one could employ oligoanionic species and make salts having appropriate ratios of quat to counterion, such as (quat)₃ citrates. These would be obvious equivalents.

Although this invention is susceptible to embodiment in many different forms, preferred embodiments of the invention are shown. It should be understood, however, that the present disclosure is to be considered as an exemplification of the principles of this invention and is not intended to limit the invention to the embodiments illustrated. It may be found upon examination that certain members of the claimed genus are not patentable to the inventors in this application. In this event, subsequent exclusions of species from the compass of applicants' claims are to be considered artifacts of patent prosecution and not reflective of the inventors' concept or description of their invention; the invention encompasses all of the members of the genus (I) that are not already in the possession of the public.

### Abbreviations

The following abbreviations and terms have the indicated meanings throughout:
Ac=acetyl
BNB=4-bromomethyl-3-nitrobenzoic acid
Boc=t-butyloxy carbonyl
BPE=2(4-biphenylyl)ethyl=

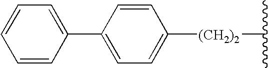

Bu=butyl
c-=cyclo
DAMGO=Tyr-ala-Gly-NMePhe-NHCH₂OH
DBU=diazabicyclo[5.4.0]undec-7-ene
DCM=dichloromethane=methylene chloride=CH₂Cl₂
DEAD=diethyl azodicarboxylate
DIC=diisopropylcarbodiimide
DIEA=N,N-diisopropylethyl amine
DMAP=4-N,N-dimethylaminopyridine
DMF=N,N-dimethylformamide
DMSO=dimethyl sulfoxide
DOR=delta opioid receptor
DPPF=1,1′-bis(diphenylphosphino)ferrocene
DVB=1,4-divinylbenzene
EEDQ=2-ethoxy-1-ethoxycarbonyl-1,2-dihydroquinoline
Fmoc=9-fluorenylmethoxycarbonyl
GC=gas chromatography
HATU=O-(7-Azabenzotriazol-1-yl)-1,1,3,3-tetramethyluronium hexafluorophosphate
HOAc=acetic acid
HOBt=hydroxybenzotriazole
KOR=kappa opioid receptor
Me=methyl
mesyl=methanesulfonyl
MOR=mu opioid receptor
MTBE=methyl t-butyl ether
NMO=N-methylmorpholine oxide
PEG=polyethylene glycol
Ph=phenyl
PhOH=phenol
NP=pentafluorophenol
PPTS=pyridinium p-toluenesulfonate
PyBroP=bromo-tris-pyrrolidino-phosphonium hexafluorophosphate
rt=room temperature
sat'd=saturated
s-=secondary
t-=tertiary
TBDMS=t-butyldimethylsilyl
TFA=trifluoroacetic acid
THF=tetrahydrofuran
TMOF=trimethyl orthoformate
TMS=trimethylsilyl
tosyl=p-toluenesulfonyl
Trt=triphenylmethyl
U69,593

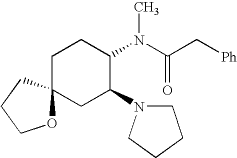

It may happen that residues in the substrate of interest require protection and deprotection during the conversion of the phenol to the desired Q. Terminology related to “protecting”, “deprotecting” and “protected” functionalities occurs throughout this application. Such terminology is well understood by persons of skill in the art and is used in the context of processes which involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is below, the person of ordinary skill can readily envision those groups that would be suitable as “protecting groups”. Suitable groups for that purpose are discussed in standard textbooks in the field of chemistry, such as Protective Groups in Organic Synthesis by T. W. Greene [John Wiley & Sons, New York, 1991], which is incorporated herein by reference.

The compounds of the invention are synthesized by one of the routes described below:

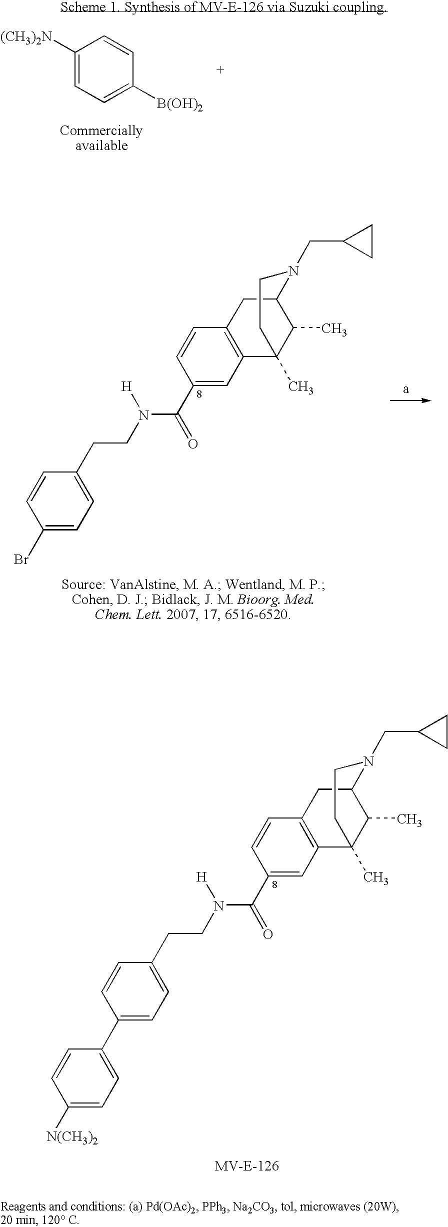

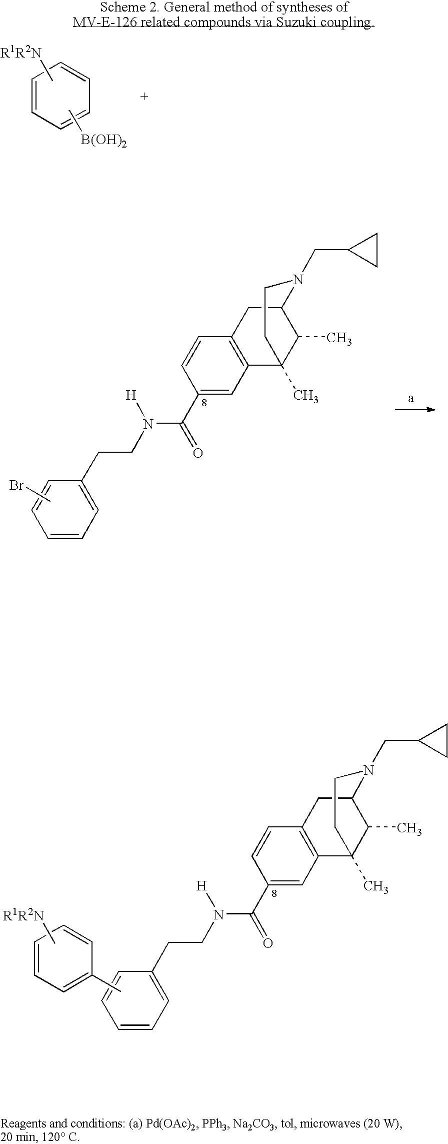

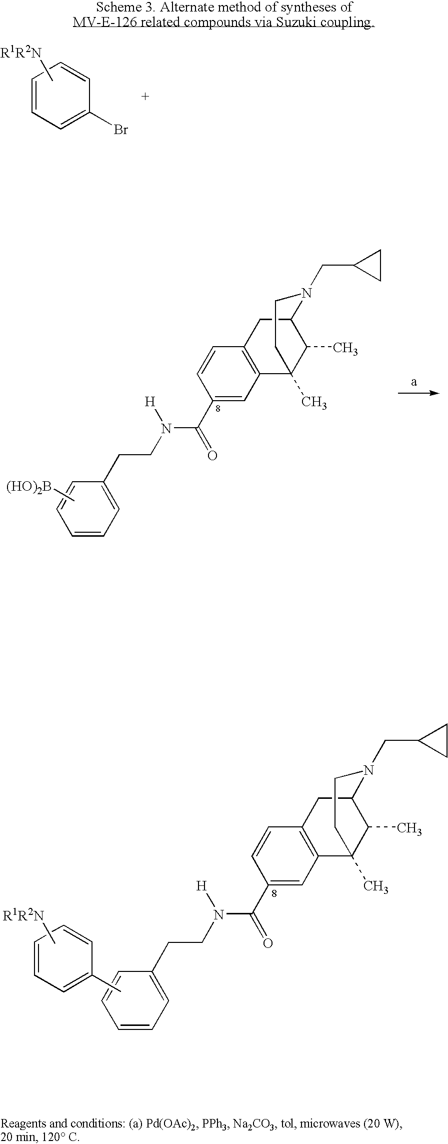

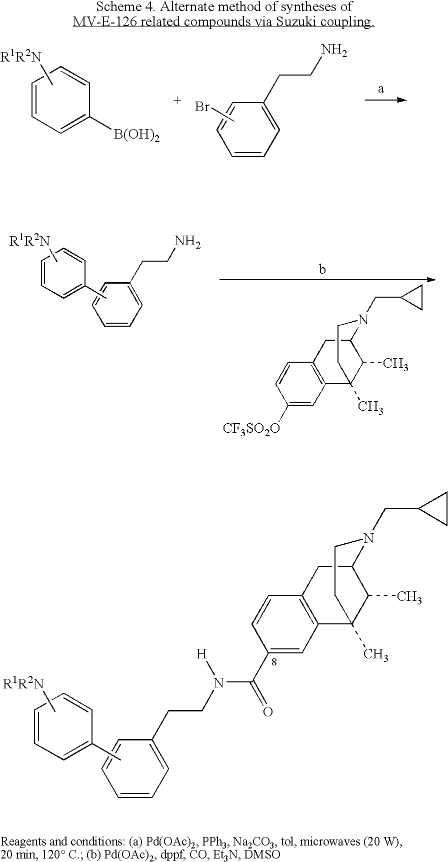

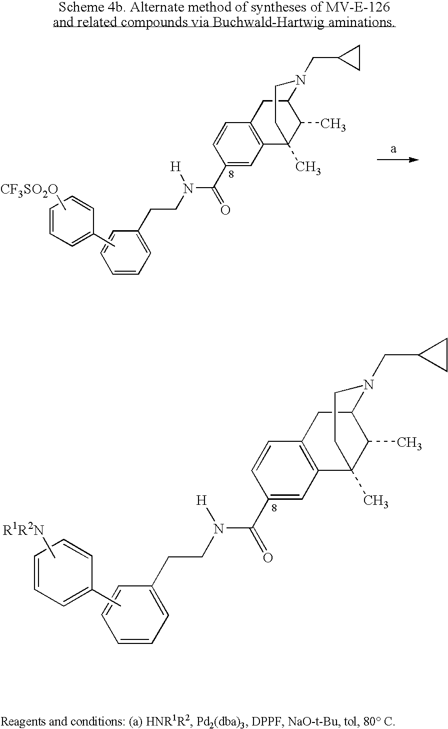

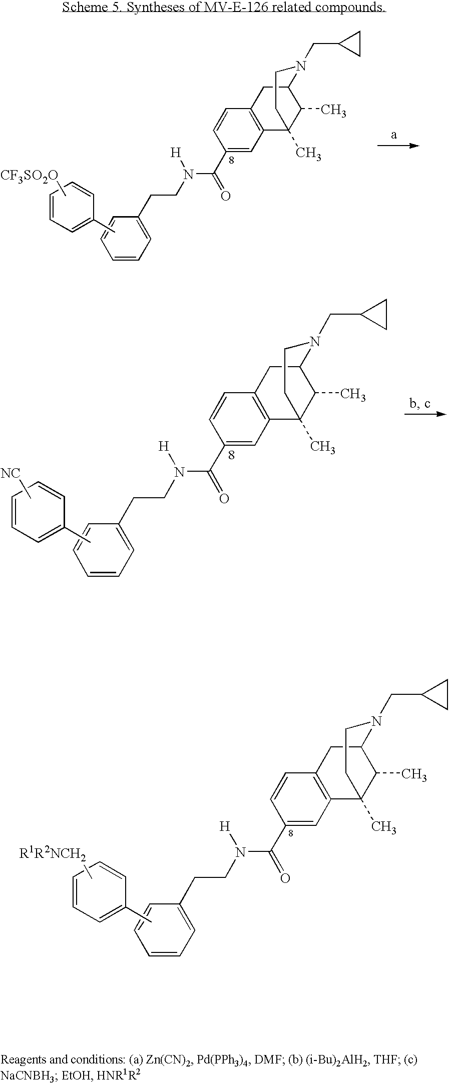

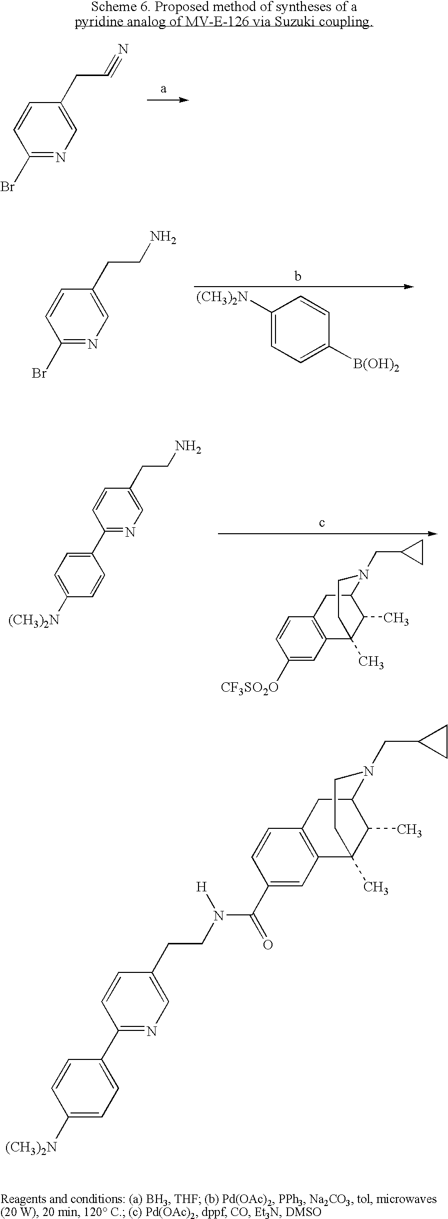

In general, the method of replacing a phenolic —OH with triflate, as shown in Scheme 4, is described in U.S. Pat. No. 6,784,187, the contents of which are incorporated herein by reference.

Proton NMR spectra and in certain cases ¹³C NMR were obtained on a Varian Unity-300 or 500 NMR spectrometer with tetramethylsilane as an internal reference for samples dissolved in CDCl₃. Samples dissolved in CD₃OD and DMSO-d₆ were referenced to the solvent. Proton NMR multiplicity data are denoted by s (singlet), d (doublet), t (triplet), q (quartet), m (multiplet), dd (doublet of doublets), and br (broad). Coupling constants are in hertz. Direct insertion probe chemical ionization mass spectral data were obtained on a Shimadzu GC-17A GC-MS mass spectrometer. Direct infusion electrospray ionization (in positively charged ion mode) mass spectral data were obtained on an Agilent 1100 series LC/MSD system (Germany). Melting points were determined on a Meltemp capillary melting point apparatus and were uncorrected. Infrared spectral data were obtained on a Perkin-Elmer Paragon 1000 FT-IR spectrophotometer. Optical rotation data was obtained from a Perkin-Elmer 241 polarimeter. The assigned structure of all test compounds and intermediates were consistent with the data. Carbon, hydrogen, and nitrogen elemental analyses for all novel targets were performed by Quantitative Technologies Inc., Whitehouse, N.J., and were within ±0.4% of theoretical values except as noted; the presence of water or other solvents was confirmed by proton NMR. Reactions were generally performed in an argon or nitrogen atmosphere. Commercially purchased chemicals were used without purification unless otherwise noted. The following reagents were purchased from Aldrich Chemical Company: N-hydroxysuccinimide, phenethylamine, 3-phenyl-1-propylamine, 4-aminobiphenyl, palladium acetate, 4-phenylbenzylamine and benzyl amine. The following reagent was purchased from Trans World Chemicals: 2-(4-biphenyl ethylamine). The following reagents were purchased from Strem Chemicals, Incorporated: 1,1′-bis(diphenyl-phosphino)ferrocene (dppf) and dichloro[1,1′-bis(diphenylphosphino)-ferrocene]palladium (II) dichloromethane adduct [PdCl₂(dppf)]. Pyridine was distilled from KOH. DMF and DMSO were distilled over CaH₂ under reduced pressure. Silica gel (Bodman Industries, ICN SiliTech 2-63 D 60A, 230-400 Mesh) was used for all flash chromatography. Amines were purchased from Aldrich Chemical Company and used as received unless otherwise indicated. Toluene and Et₂O were distilled from sodium metal. THF was distilled from sodium/benzophenone ketyl. Pyridine was distilled from KOH. Methylene chloride was distilled from CaH₂. DMF and DMSO were distilled from CaH₂ under reduced pressure. Methanol was dried over 3± molecular sieves prior to use. Silica gel (Bodman Industries, ICN SiliTech 2-63 D 60A, 230-400 Mesh) was used for flash column chromatography.

In general, the chemistry described above works in the presence of the variety of functional groups found on known core structures. The exceptions would be morphine and congeners having a free 6-OH, which can be protected by a TBDPS (t-butyldiphenylsilyl) group [see Wentland et al., “Selective Protection and Functionalization of Morphine . . . ”, J. Med. Chem. 43, 3558-3565 (2000)].

I claim:

1. A compound of formula I: 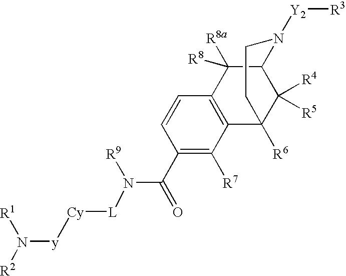
wherein
R¹ and R² are each independently chosen from hydrogen, optionally substituted lower alkyl, optionally substituted alkenyl, optionally substituted alkynyl, optionally substituted aryl, —COR¹⁰, —SO₂R¹⁰, —CONR¹⁰R¹¹, —C(═S)R¹⁰, —C(═NOR¹¹)R¹⁰, C(═NR¹⁰)R¹¹, and —SO₂NR¹⁰R¹¹;
or, taken together with the nitrogen to which they are attached, R¹ and R² may form from one to three rings, said rings having optional additional substitution;
R³ is chosen from hydrogen, C₁-C₈ hydrocarbon, heterocyclyl, aryl and hydroxyalkyl;
R⁴ is chosen from hydrogen, hydroxyl, amino, lower alkoxy, C₁-C₂₀ alkyl and C₁-C₂₀ alkyl substituted with hydroxyl or carbonyl;
R⁵ is lower alkyl;
R⁶ is lower alkyl;
R⁷ is chosen from hydrogen, NR¹⁰R¹¹ and —OR¹⁰; or
together R⁴, R⁵, R⁶ and R⁷ may form from one to three rings, said rings having optional additional substitution;
R⁸ and R^{8a} are both hydrogen or taken together R⁸ and R^{8a} are ═O;
R⁹ is chosen from hydrogen and lower alkyl;
R¹⁰ and R¹¹ are each independently hydrogen, optionally substituted lower alkyl, optionally substituted alkenyl, optionally substituted alkynyl, optionally substituted aryl, hydroxyl, amino or optionally substituted lower alkoxy;
y is —(C(R¹⁰)(R¹¹))p- or a direct bond, wherein p is 0, 1, 2, 3, 4, 5, 6, or 7;
Y₂ is a direct bond or —(C(R¹⁰)(R¹¹))q-, wherein q is 0, 1, 2, 3, 4 or 5;
L is a direct bond or —(C(R¹⁰)(R¹¹))q-; and
Cy is Ar¹—B—Ar², wherein
Ar¹ is absent, or an aryl or heteroaryl radical having from 1 to 4 N, O and/or S atoms, which may be unsubstituted or mono-, di- or trisubstituted by halogen, lower alkyl, alkenyl, alkynyl, cycloalkyl, —OR¹⁰, —NR¹⁰R¹¹, —CN, —COR¹⁰ or —COOR¹⁰;
B is a direct bond, —O—, —NR¹⁰, —SO₂, or —(C(R¹⁰)(R¹¹)s-, wherein s is 0, 1, 2, 3, 4 or 5; and
Ar² is aryl or heteroaryl radical having from 1 to 4 N, O and/or S atoms, which may be unsubstituted or mono-, di- or trisubstituted by halogen, lower alkyl, alkenyl, alkynyl, cycloalkyl, —OR¹⁰, —NR¹⁰R¹¹, —COR¹⁰ or —COOR¹⁰.

2. A compound of claim 1 wherein Cy is selected from: 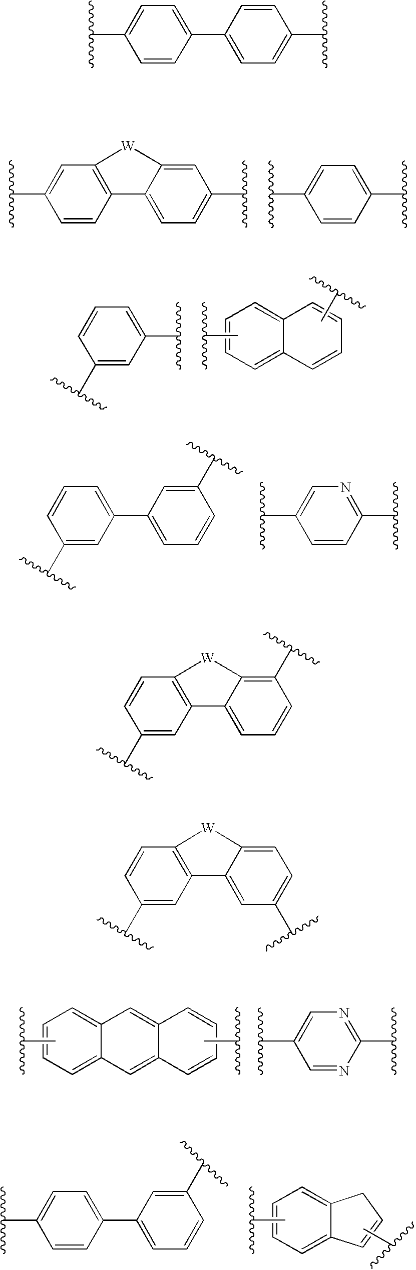 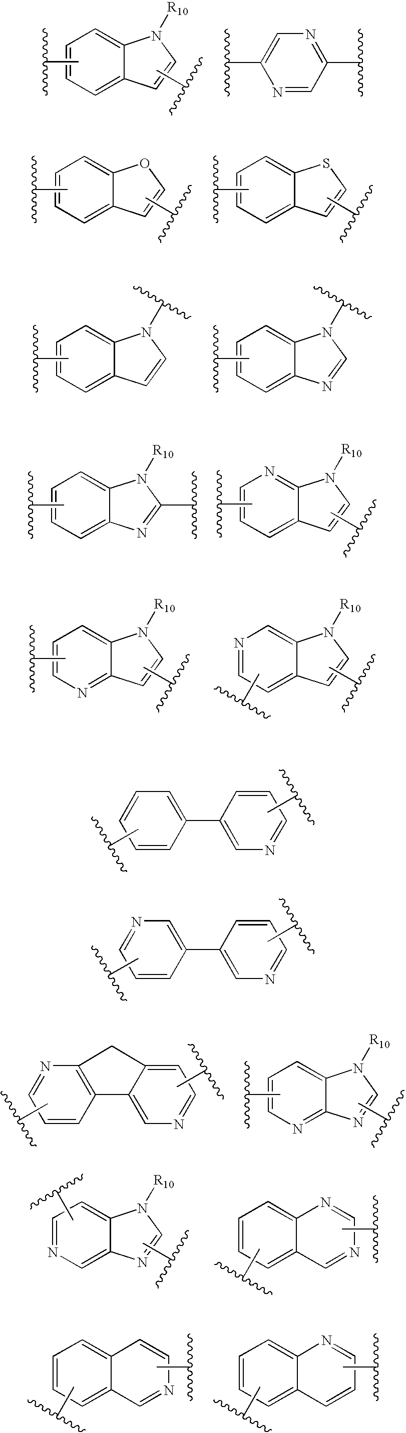 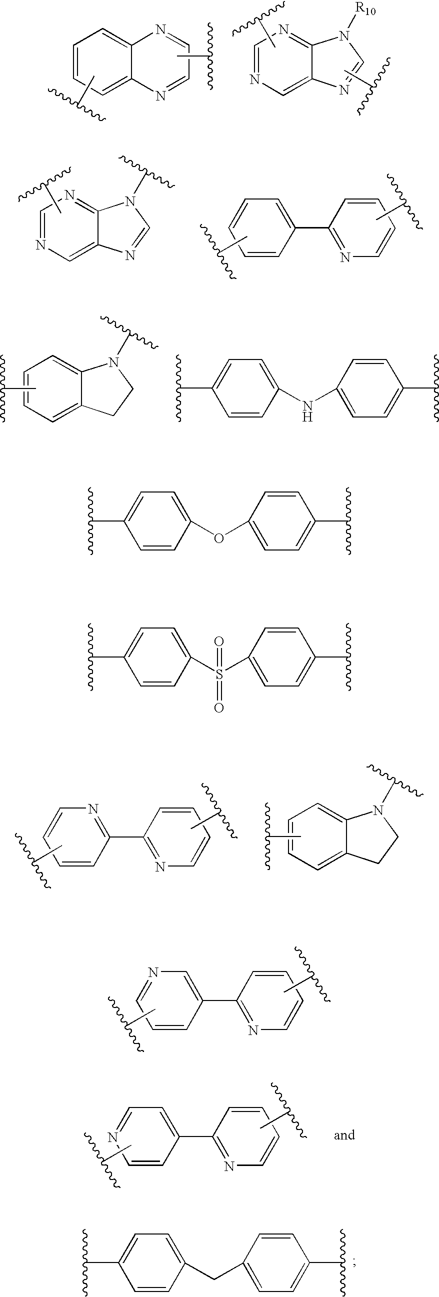
W is selected from [C(R⁹)₂]ₙ, CR⁸R^{8a}, O, NR⁹, S and CR⁹═CR⁹; and
n is 1, 2, 3, 4 or 5.

3. A compound of claim 1 of formula II: 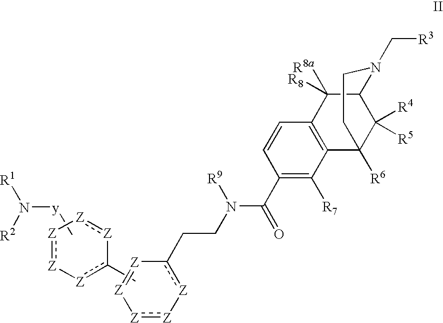
wherein
Z is CR¹⁰ or N, with the proviso that,
at the points of attachment of the NR¹R²y group to the distal aromatic ring and of the distal aromatic ring to the proximal aromatic ring, Z must be C.

4. A compound according to claim 3 of formula

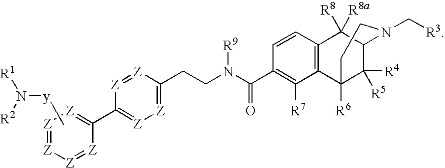

5. A compound according to claim 4 of formula

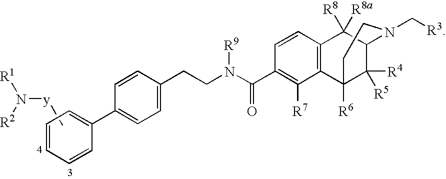

6. A compound according to claim 1 wherein:
R³ is chosen from hydrogen, cyclopropyl, cyclobutyl, phenyl, vinyl, dimethylvinyl, hydroxycyclopropyl, furanyl, and tetrahydrofuranyl;
R⁴ is chosen from hydrogen and 3-oxo-5-cyclopentyl-1-pentanyl;
R⁵ is methyl;
R⁶ is methyl or ethyl;
R⁸ and R^{8a} are both hydrogen; and
R⁹ is hydrogen.

7. A compound according to claim 6 wherein -yNR¹R² is substituted at the 4-position.

8. A compound according to claim 7 wherein y is a direct bond.

9. A compound according to claim 8 wherein R¹ and R² are each selected from methyl and hydrogen.

10. A compound according to claim 8 wherein R¹ is hydrogen, R² is selected from substituted alkyl, —SO₂R¹⁰ and —COR¹⁰, and R¹⁰ is selected from optionally substituted lower alkoxy, optionally substituted lower alkyl and optionally substituted aryl.

11. A compound according to claim 10 wherein R² is, together with the nitrogen to which it is attached, fluorenylmethyl carbamate, tert-butyl carbamate, benzyl carbamate, acetamide, trifluoroacetamide, benzylamine, triphenylmethylamine or toluenesulfonamide.

12. A compound according to claim 7 wherein y is CH₂.

13. A compound according to claim 12 wherein R¹ and R² are each selected from methyl and hydrogen.

14. A compound according to claim 12 wherein R¹ is hydrogen, R² is selected from substituted alkyl, —SO₂R¹⁰ and —COR¹⁰, and R¹⁰ is selected from optionally substituted lower alkoxy, optionally substituted lower alkyl and optionally substituted aryl.

15. A compound according to claim 14 wherein R² is, together with the nitrogen to which it is attached, fluorenylmethyl carbamate, tert-butyl carbamate, benzyl carbamate, acetamide, trifluoroacetamide, benzylamine, triphenylmethylamine or toluenesulfonamide.

16. A compound according to claim 6 wherein -yNR¹R² is substituted at the 3-position.

17. A compound according to claim 16 wherein y is a direct bond.

18. A compound according to claim 17 wherein R¹ and R² are each selected from methyl and hydrogen.

19. A compound according to claim 17 wherein R¹ is hydrogen, R² is selected from substituted alkyl, —SO₂R¹⁰ and —COR¹⁰, and R¹⁰ is selected from optionally substituted lower alkoxy, optionally substituted lower alkyl and optionally substituted aryl.

20. A compound according to claim 19 wherein R² is, together with the nitrogen to which it is attached, fluorenylmethyl carbamate, tert-butyl carbamate, benzyl carbamate, acetamide, trifluoroacetamide, benzylamine, triphenylmethylamine or toluenesulfonamide.

21. A compound according to claim 9 of formula 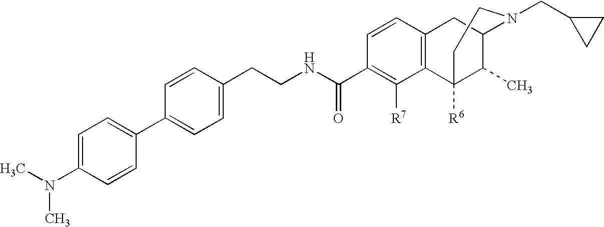
wherein R⁶ is methyl or ethyl.

22. A compound according to claim 1 wherein together R⁵ and R⁶ form one ring, said compound having the structure:

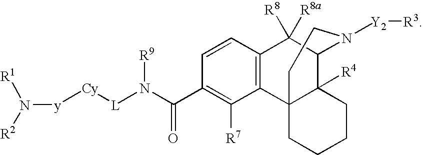

23. A compound according to claim 22 wherein
R⁸ and R^{8a} are hydrogen;
R³ is chosen from hydrogen, cyclopropyl, cyclobutyl, vinyl and tetrahydrofuranyl; and
R⁴ is hydrogen, hydroxyl or amino.

24. A compound according to claim 1 wherein together R⁵, R⁶ and R⁷ form two rings, having the structure: 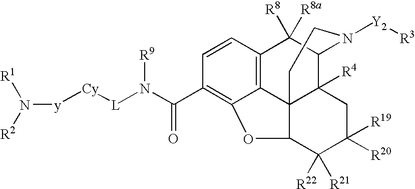
wherein
R⁴ is hydrogen, hydroxy, amino or lower alkoxy;
R¹⁹ is hydrogen or lower alkyl;
R²⁰ is chosen from hydrogen, lower alkyl and hydroxy(lower alkyl); or together, R¹⁹ and R²⁰ form a spiro-fused carbocycle of 5 to 10 carbons;
R²¹ is hydrogen;
R²² is chosen from hydroxy, lower alkoxy and —NR¹³R¹⁴; or
together, R²¹ and R²² form a carbonyl or a vinyl substituent; or
together, R⁴ and R²¹ form a sixth ring;
R¹³ is hydrogen or optionally substituted lower alkoxy; and
R¹⁴ is hydrogen, optionally substituted lower alkoxy, acyl or fumarate.

25. A compound according to claim 24, wherein together, R⁴ and R²¹ form a sixth ring, of formula:

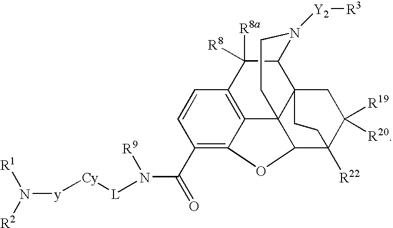

26. A compound according to claim 24, wherein R⁴ and R²¹ form a sixth ring, of formula 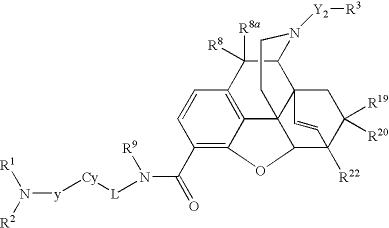
wherein
R¹⁹ is hydrogen;
R²⁰ is hydroxy(lower alkyl); and
R²² is lower alkoxy.

27. A compound according to claim 22 wherein 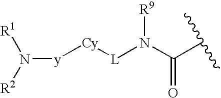
is represented by

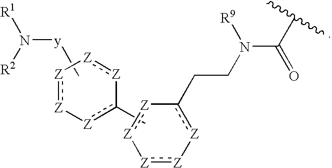

28. A method for preparing a second compound that interacts with an opioid receptor when a first compound that interacts with an opioid receptor is known, said first compound containing a phenolic hydroxyl, said method comprising converting said phenolic hydroxyl to a residue of formula:

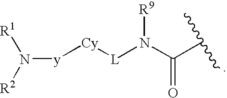

29. A method according to claim 28 wherein the residue 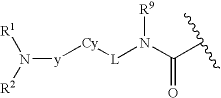
is 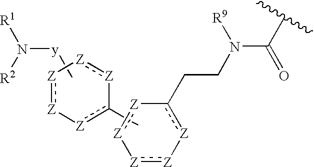
wherein
Z is CR¹⁰ or N, with the proviso that, at the points of attachment of the NR¹R²y group to the distal aromatic ring and of the distal aromatic ring to the proximal aromatic ring, Z must be C.

30. A pharmaceutical formulation comprising a compound according to claim 1 and a pharmaceutically acceptable carrier.

31. A method of preventing or treating a condition or disease associated with binding opioid receptors in a patient in need thereof, comprising the step of administering to said patient a composition comprising an effective amount of a compound according to claim 1.

32. A method according to claim 31 wherein said disease or condition is chosen from the group consisting of pain, pruritis, diarrhea, irritable bowel syndrome, gastrointestinal motility disorder, obesity, respiratory depression, convulsions, coughing, hyperalgesia and drug addiction.

33. A method according to claim 32 wherein said drug addiction is selected from heroin, cocaine, nicotine and alcohol addiction.

34. A method according to claim 32, wherein the condition is pain and the composition further comprises an effective amount of an opioid.
